(19) United States
(12) Patent Application Publication
(10) Pub. No.: US 2026/0233017 A1
(43) Pub. Date: Aug. 13, 2026

(54) METHOD AND APPARATUS FOR THE ANTI-INFLAMMATORY TREATMENT OF ATHEROSCLEROSIS AND CORONARY ARTERY DISEASE

(71) Applicant: Victor I. CHORNENKY, Minnetonka, MN (US)

(72) Inventor: Victor I. CHORNENKY, Minnetonka, MN (US)

(21) Appl. No.: 19/531,396

(22) Filed: Feb. 5, 2026

Related U.S. Application Data
(60) Provisional application No. 63/757,275, filed on Feb. 11, 2025.

(51) Int. Cl. A61N 1/40

(52) U.S. Cl. CPC A61N 1/40

(57) ABSTRACT

A treatment method for atherosclerosis includes activating adenosine receptors A2aAR in immune and parenchymal cells to promote the inflammatory process in atherosclerotic lesions. Activation is accomplished by an electrical component of a pulsed electromagnetic field (PEMF) applied locally to the affected segment of an artery or to the entire heart. Activation of the electric field in the treatment zone may be applied in several spatial directions. A treatment apparatus includes an electronic controller and an electromagnetic applicator. The controller supplies pulsed voltage to the applicator, which in turn produces a stimulating electric field within the treatment zone. The apparatus includes a set of parallel straight wires placed on the patient's chest to generate a linear stimulating electric field. Additional sets of parallel wires can be used to produce multidirectional stimulation of the heart. The applicator is free from “dead” zones and provides highly uniform electric field.

### PRIORITY

This application claims the priority benefit of U.S. Provisional Application No. 63/757,275, filed on February 11, 2025, which is hereby incorporated herein by reference in its entirety.

### FIELD

The invention relates to a method and apparatus for the anti-inflammatory treatment of atherosclerosis in general and coronary artery disease (CAD) in particular.

### BACKGROUND

Atherosclerosis is a progressive disease characterized by the buildup of plaque within the walls of arteries. This process involves several stages:
1. Endothelial dysfunction: the earliest stage of atherosclerosis involves damage or dysfunction of the endothelium, the inner lining of blood vessels. Endothelial cells lose their ability to regulate blood vessel dilation, blood clotting, and immune function. This leads to an increased permeability of the vessel wall to lipids.
2. Lipoprotein deposition: low-density lipoproteins (LDL) infiltrate the damaged endothelium and deposit within the arterial wall. Under conditions of inflammation LDL particles become oxidized and are engulfed by macrophages, forming foam cells, which are the hallmark of early atherosclerotic lesions.
3. Fatty streak formation: the accumulation of foam cells leads to the formation of fatty streaks, the first visible signs of atherosclerosis. The fatty streaks appear as yellowish streaks along the arterial wall and consist of lipid-laden macrophages and smooth muscle cells.
4. Plaque formation: fatty streaks develop into more complex atherosclerotic plaques as more lipids, inflammatory cells, and smooth muscle cells accumulate. Plaques are composed of a lipid core, which includes cholesterol, necrotic cellular debris, and a fibrous cap made of collagen and smooth muscle cells.
Plaques are classified into two main types: stable and unstable, depending on their composition and the risk they pose for causing cardiovascular events like heart attacks or strokes.
5. Stable atherosclerotic plaque: stable plaques have a thick fibrous cap that covers the lipid core. The caps contain smooth muscle cells and collagen, which make the plaque more robust and less likely to rupture. These plaques tend to grow slowly and gradually obstruct blood flow over time, leading to chronic conditions such as stable angina (chest pain). Because of their thick cap, they are less prone to rupture and causing heart attacks.
Stable plaques are presented with chronic, predictable symptoms like stable angina. They can be managed with lifestyle changes, medications, and, if they cause significant blood flow restriction, procedures like angioplasty.
6. Unstable atherosclerotic plaque: unstable plaques have a thin, fragile, fibrous cap and a large lipid-rich core. They are often infiltrated with macrophages, which produce metalloprotease, weakening the cap further. These plaques are prone to rupture. When the thin cap breaks, a highly thrombogenic lipid core is exposed to the bloodstream, leading to the formation of a blood clot (thrombus). The clot can suddenly block the artery and cause an acute event like a heart attack.
Unstable plaques are responsible for acute coronary syndromes, including unstable angina and myocardial infarction (heart attack). They require urgent medical attention and are typically treated with antiplatelet drugs, anticoagulants, and sometimes emergency procedures like angioplasty or coronary artery bypass grafting (CABG).

In summary, stable plaques in coronaries cause more chronic, predictable issues, while unstable plaques are more dangerous due to their potential to rupture and cause sudden life-threatening events.

Inflammation is intricately involved in all stages of atherosclerosis, from the initiation of atherosclerosis to the complications of plaque rupture. According to the latest evidence, the stable plaques are associated with a slow growth under conditions of low-grade inflammation, whereas the unstable plaques are associated with a rapid growth under conditions of high-grade inflammation.

### Reversed Cholesterol Transport (RCT)

Accumulation of lipids in arterial plaques is a dynamic metabolic process that is defined not only by the deposition of LDL in the artery walls but also by a process called reversed cholesterol transport (RCT) which clears these deposits. RCT is a crucial physiological process in which excess cholesterol is removed from peripheral tissues (like arterial walls) and transported back to the liver for excretion.

Cholesterol is converted in the liver into bile acids and excreted into the intestines via bile. It can also be directly excreted into the bile as free cholesterol. This final step removes cholesterol from the body, completing the reverse cholesterol transport process.

The effectiveness of RCT is crucial in reducing the risk of atherosclerosis, as it helps clear cholesterol from arterial plaques and maintain cholesterol homeostasis in the body.

Inflammation impairs RCT by disrupting key steps of the RCT process:
a. Inflammation affects macrophages in atherosclerotic plaques by reducing their ability to efflux cholesterol (remove it) via transporters like ABCA1 and ABCG1. This leads to the accumulation of cholesterol within macrophages and promotes plaque growth.
b. Inflammation reduces the levels and functionality of high-density lipoprotein (HDL), which helps remove cholesterol from macrophages (foam cells) in plaques.
c. Inflammation impairs liver receptors and processes, such as the uptake of cholesterol from HDL, reducing the liver's capacity to metabolize and excrete cholesterol.

Overall, inflammation impairs multiple components of the reverse cholesterol transport pathway, contributing to the progression of atherosclerosis and reducing the clearance of cholesterol from plaques.

### Inflammation And Plaque Evolution

Recent serial intravascular imaging studies have shown that atherosclerotic lesions in human coronary arteries progress over time from a stable to an unstable state and vice versa, from unstable to a stable state. Rapid progression of lesions to a dangerously unstable state occurs with high-grade inflammation, whereas regression, reduction in total volume and stabilization occur with low-grade inflammation.

This new understanding of the atherosclerotic plaque evolution suggests a new approach to the treatment of unstable atherosclerotic plaque: converting unstable lesions into stable, non-life-threatening lesions using anti-inflammatory therapy.

Currently, there is no effective anti-inflammatory therapy for the treatment of atherosclerosis. Regular anti-inflammatory treatment with NSAIDs and/or steroids is not advisable due to serious side effects of such drugs.

Statins, drugs used to lower cholesterol, have been found to have anti-inflammatory properties that are useful for patients with atherosclerosis. However, statins have serious side effects and have relatively little anti-inflammatory activity, which prevents their use with high doses for intensive anti-inflammatory therapy.

Recently proposed anti-cytokine therapy based on blocking the main pro-inflammatory cytokines such as TNF-α, IL-1 and others keep some promises but so far has had limited success.

The main conceptual weakness of the anti-cytokine approach is that cardiovascular inflammation is a very complex and well-coordinated process with dozens of participants: multiple cells of innate and adaptive immune systems, epithelial cells, smooth muscle cells, cardiomyocytes, various cytokines, etc. It seems unlikely that disabling one or two cytokines will substantially and without side effects reduce the overall inflammation in the arteries.

It would be highly desirable to find a way to downregulate the entire inflammatory process with one action, similar to what happens in nature – by activating a recently discovered adenosine-A2aAR anti-inflammatory pathway in all cells involved in the inflammatory process.

There is a realistic hope for finding adequate anti-inflammatory treatment for atherosclerosis. This hope is associated with aforementioned adenosine-A2aAR signaling pathway.

### Adenosine Anti-Inflammatory Pathway

Adenosine, a purine nucleoside generated by metabolically stressed and inflamed tissues, is recognized as a major endogenous anti-inflammatory regulator. Under normal conditions, adenosine is continuously released from cells as a product of ATP degradation. Adenosine concentration in the extracellular space is controlled by an enzyme adenosine deaminase (ADA) which breaks it down and keeps the concentration level in high-nanomolar to low-micromolar range. However, during inflammation levels of extracellular adenosine rise dramatically (up to 200-fold).

Adenosine regulates the function of the innate and adaptive immune systems through targeting virtually every cell type involved in orchestrating the immune/inflammatory response. Of the four adenosine receptors (A1, A2a, A2b, A3), A2a receptors have taken center stage as the primary anti-inflammatory effectors of extracellular adenosine. The broad anti-inflammatory effect of A2a receptor activation is a result of the predominant expression of A2a receptors in every immune cell.

A2a receptor activation inhibits early and late inflammatory events occurring during an immune response, which include immune cell trafficking, proliferation, proinflammatory cytokine production, and cytotoxicity. A2aARs modulation of immune cell function and inflammatory signaling pathways shifts the balance from pro-inflammatory to anti-inflammatory responses, thereby playing a crucial role in limitation of tissue damage and facilitation of tissue restoration.

As far as atherosclerosis is concerned, A2a receptors are found in all cells participating in inflammation of atherosclerotic lesions: macrophages, various immune cells of innate and adaptive immune systems, smooth muscle cells and vascular endothelium.

Macrophages play a critical role in immune response. They start and maintain inflammation by releasing cytokines stimulating the immune system and play an important anti-inflammatory role during the late stage of inflammation. Macrophages that encourage inflammation are called M1 macrophages, whereas those that decrease inflammation and promote tissue restoration are called M2 macrophages. Macrophages morph into phenotypes M1 or M2 depending on the environment in which they are activated. In presence of cytokines such as IL-12 and IL-23 in initial stage of immune response, the “classically” activated macrophages are pro-inflammatory type M1, whereas at the final stage of inflammation, in presence of cytokines IL-10, macrophages become “alternatively” activated into anti-inflammatory type M2 that promotes tissue healing and restoration.

Consistent with its generally restorative function in tissues, A2a receptor activation has been repeatedly shown to have effects that prevent excessive classical macrophage activation thereby resulting in tissue protection. Besides the suppressive effect of adenosine on the production of proinflammatory mediators, adenosine enhances production of anti-inflammatory cytokine IL-10 that promotes activation of macrophages into type M2 participating in tissue restoration.

The discovery of the anti-inflammatory Adenosine – A2aAR pathway was met by clinicians and pharmacologists with great optimism: it seemed to promise the effective treatment for many inflammatory diseases. Pfizer and other pharmaceutical companies launched clinical trials for treating lung inflammation with newly developed adenosine agonists.

Regretfully, the human trials were discontinued because of strong hemodynamic side effects. At concentrations effective in suppression of inflammation, adenosine agonists caused life-threatening drops in blood pressure and cardiac index. This hemodynamic side effect is a consequence of the ability of adenosine-A2aARs pathway to produce vasodilatory effects. At high systemic concentrations of adenosine agonists, A2aARs of vascular endothelium dilate the whole vascular bed and cause life-threatening complications that make systemic use of adenosine agonists unacceptable.

Contrary to lessons learned from the Pfizer study, A2aAR adenosine receptors are known to participate in regulating different functions in various organs without any side effects. It has a simple explanation: adenosine in plasma has a very short half-life (10 seconds) which limits its effects to tissues where it was produced, whereas adenosine agonists have half-life of 5-9 hours and act systemically, causing harmful hemodynamic effects.

The ultimate question about the possibility of using adenosine – A2aAR pathway for anti-inflammatory applications is: can it be activated locally, without creating systemic hemodynamic side effects?

The present invention suggests that the answer is YES. This answer has come from a special form of physical therapy - Pulsed Electromagnetic Field (PEMF) stimulation.

### Pulsed Electromagnetic Field Stimulation And Adenosine

Recently it has been established by Dr. Varani et al. that the anti-inflammatory action of PEMF stimulation is attributable to its ability to increase the expression of adenosine receptors A2aAR on cellular membranes. According to their data, PEMF stimulation translocates A2 receptors from stand-by positions in cytosol onto the cellular membranes where they become active and available for binding with adenosine ligands.

When ligands bind to receptors, the intensity of the cellular response depends on both the concentration of ligands in the extracellular space and the concentration of receptors on the cellular membranes. As a result, the same response can be achieved by two different ways: by changing the concentration of adenosine around the cell or by changing concentration of receptors on the cellular membrane.

The essence of Dr. Varani's discovery is that PEMF stimulation can activate the adenosine-A2aAR anti-inflammatory pathway locally by elevating expression of A2a receptors in the treatment zone only, without an increase of systemic concentration of adenosine, thereby avoiding hemodynamic side effects.

It should be mentioned that the active agent of PEMF is the electric field (EF), not the magnetic field. The magnetic component of PEMF practically does not interact with biological tissues and is used only as a carrier that deeply penetrates in tissues and a change of which generates EF (Faraday effect). In this regard, the intensity of PEMF stimulation is measured by the amplitude of the EF pulses.

According to experimental data, expression of A2aAR receptors can be significantly upregulated by pulsed electric fields with amplitudes greater than 50 microVolt/cm. In an environment rich in extracellular adenosine, which is always the case with inflamed or stressed tissues, the upregulation of expression of A2aAR receptors leads to proportional amplification of adenosine – A2aAR signaling. In other words, in the treatment zone PEMF stimulation triggers the same anti-inflammatory response of the cells as a local increase in concentration of adenosine, or adenosine agonists, in the extracellular space. In either case the amplitude of signals from A2aAR receptors to cellular machinery increases as well as the downstream effects of A2aAR signaling.

PEMF stimulation of A2aAR receptors provides a novel powerful therapeutic tool for treatment of various immune/inflammatory diseases. Amplified by PEMF stimulation, A2aAR signaling inhibits cytokine-producing activity in immune and parenchymal cells, suppresses chronic inflammation and restores damaged tissues. Discovered and understood only in the recent decades, the adenosine A2aAR signaling pathway is one of the most powerful anti-inflammatory regulators discovered to date. Several well conducted clinical studies demonstrated that activation of A2aAR receptors leads to suppression of inflammation and promotes restoration of different damaged tissues.

It is instructive to keep in mind that the Adenosine-A2aAR anti-inflammatory pathway in joints is activated via induction of electric field, similar to PEMF stimulation. In the case of joints, though, the electric field is generated not by a change in the magnetic field as in Faraday effect, but due to a piezoelectric effect, which generates an electric field in bones and cartilage in response to mechanical stress applied to the joint. The electric field translocates A2aAR receptors onto the cell membranes of chondrocytes (cartilage cells), which increases their expression and enhances the anti-inflammatory signal from extracellular adenosine. In both cases, the main effector of the anti-inflammatory action is the electric field.

A2aAR receptors are sensitive to an electric field and under the field action are able to relocate from a stand-by position in cytosol onto the cellular membranes. This phenomenon enables control of A2aAR receptor expression and its anti-inflammatory effects using an external electromagnetic field, forming the scientific basis for anti-inflammatory PEMF therapy.

There is an ongoing need to develop effective anti-inflammatory therapies that could treat various forms of immune/inflammatory diseases including arteriosclerosis, coronary artery disease, heart failure and other heart conditions.

### SUMMARY

The disclosed method of treatment of atherosclerosis offers a new approach to the long-standing problem of anti-inflammatory treatment of atherosclerosis and coronary artery disease (CAD). The treatment method in certain embodiments involves application of pulsed electromagnetic field stimulation (PEMF) to a treatment zone that increases the expression of A2aAR adenosine receptors in immune and parenchymal cells involved in the inflammatory process. Binding increased numbers of receptors to adenosine ligands enhances the activity of the adenosine-A2aAR pathway, whose primary function is to downregulate immune system activity, reduce inflammation, and promote tissue restoration.

The treatment method in certain examples includes activating adenosine receptors A2aAR in immune and parenchymal cells that promote the inflammatory process in atherosclerotic lesions. Activation is accomplished by an electrical component of a PEMF applied locally to the affected segment of an artery or, in the case of CAD, to the entire heart. To maximize A2aAR activation the electric field in the treatment zone may be applied in several spatial directions. Activated A2aARs create a non-inflammatory environment that restores the full function of the Reverse Cholesterol Transport (RCT) inhibited by inflammation. RCT removes cholesterol from atherosclerotic plaques, thereby promoting the reversal of atherosclerosis.

Also provided is an apparatus for the anti-inflammatory treatment of atherosclerosis, and in particular, CAD. The apparatus includes an electronic controller and an electromagnetic applicator. The controller supplies pulsed voltage to the applicator, which in turn produces a stimulating electric field within the treatment zone surrounding the heart. Unlike traditional coil-based cylindrical applicators, the apparatus comprises a set of parallel straight wires placed on the patient's chest and generating a linear stimulating electric field. Additional sets of parallel wires can be used to produce multidirectional stimulation of the heart. The applicator is free from producing “dead” zones within the treatment zone and provides a highly uniform electric field.

The main objective of the present invention is to provide a non-invasive method for treatment of atherosclerosis, in particular in CAD, as well as an apparatus for delivering the treatment.

Another objective is to restore the normal efficiency of Reversed Cholesterol Transport inhibited by inflammation.

Yet another objective is to minimize or stop dangerous development of atherosclerotic lesions in coronary arteries and convert the life-threatening unstable plaques into stable ones that can be managed by medications.

Yet another objective is to decrease the overall plaque burden in coronaries and improve coronary blood flow.

Yet another objective is to maintain the heart health through preventive use of the present invention to counteract diabetic and age-related systemic inflammation and prolong life of the users.

The disclosed apparatus for delivery anti-inflammation cardiovascular therapy comprises a novel electromagnetic (PEMF) applicator and an electronic controller that supplies the applicator with pulse power and controls the treatment procedure.

For adequate stimulation of the heart, EF stimulation must be uniform throughout the treatment zone both in the XY planes parallel to the plane of patient’s chest and along the Z axis perpendicular to it. Here, the X, Y, Z axes correspond to the width, height and depth of the treatment zone. The axes could be labeled differently as well.

Electromagnetic coils commonly used for PEMF stimulation do not provide uniform EF. It is a widely spread misconception that a uniform magnetic field created by a simple cylindrical coil creates a uniform electric field. In reality, EF generated by a simple cylindrical coil is highly nonuniform: it has a zero value at the coil’s center along its axis and increases to a maximum value at the coil’s radius near the wires with current. In this configuration, the EF stimulation has a “dead zone” around the coil’s central axis in which the EF amplitude is zero or below the required therapeutic minimum and is unable to provide adequate treatment within the treatment zone. This is a significant drawback of existing PEMF applicators.

The electromagnetic applicator disclosed herein does not have “dead zones” and meets the requirements for the desired EF uniformity in the treatment zone. The new applicator disclosed herein includes a set of parallel straight wires positioned in one plane and configured for placement in close proximity to the treatment area (for example, on the patient's chest). The set of parallel wires defines a treatment zone with the heart at its center.

The detailed description of the therapy and preferred embodiments for the invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### BRIEF DISCRIPTION OF THE DRAWINGS

FIG. 1 is a schematic representation of the new stimulation applicator for ant-inflammatory treatment of the heart.

FIG. 2 is a schematic illustration of the new applicator comprising two stimulation coils crossed at 90 degrees to each other and providing two-dimensional PEMF stimulation of the heart.

FIG. 3 is a diagram that illustrates the time diagrams of voltage applied to the applicators (a), electric current in the coils and magnetic field in the treatment zone (b) and stimulating electric field in the treatment zone (c).

FIG. 4 is a diagram presenting a time diagram of electric pulses applied in directions of axes X and Y of the treatment zone.

### DETAILED DESCRIPTION

In the following descriptions, the present invention will be explained with reference to various example embodiments; nevertheless, these example embodiments are not intended to limit the present invention to any specific example, embodiment, environment, application, or implementation described therein. Therefore, descriptions of these example embodiments are only provided for purpose illustration rather than to limit the present invention.

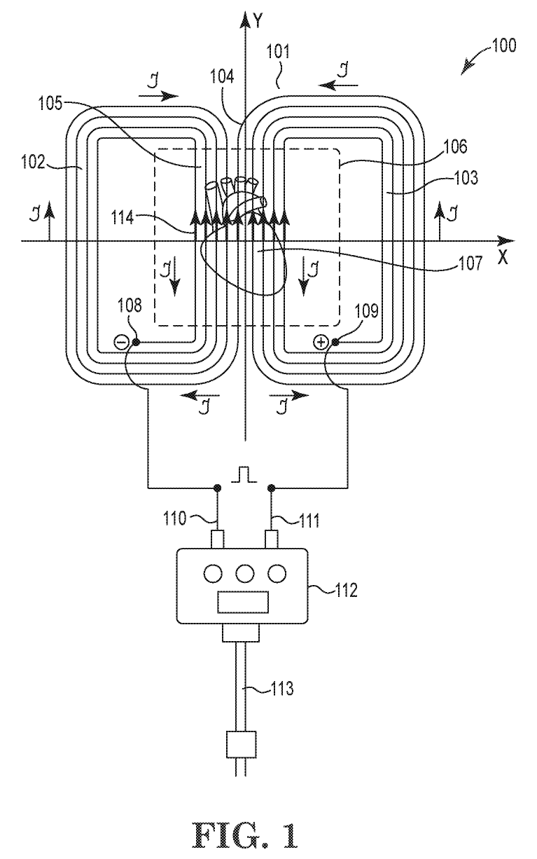

An apparatus 100 for local treatment of atherosclerosis is depicted in FIG. 1. It comprises electromagnetic applicator 101, that includes left coil 102 and right coil 103 connected to each other with wire 104, common for both coils and located at the center of applicator 101. In the central area of applicator 101, coils 102 and 103 form a set of parallel wires 105 that provides electric field stimulation in the treatment zone 106, outlined by the dashed line. The target of stimulation, heart 107, is in the center of treatment zone 106. Terminals 108 and 109 of applicator 101 are connected to the output electrodes 110 and 111 of computerized controller 112, which supplies electrical pulses to applicator 101 during operation. Power cable 113 provides controller 112 with power and is connected to a power grid or other power source (e.g. a battery).

FIG. 1 depicts applicator 101 in which left coil 102 and right coil 103 are connected in series to each other with wire 104 and connected to electrodes 108 and 109. In another version of the invention, the wire 104 can be cut in two with its left end connected to electrode 109, whereas the right end is connected to electrode 108. In this configuration, the left and the right coils are connected to electrodes 108 and 109 in parallel.

The controller 112 provides applicator 101 with trains of positive or negative electric pulses to create electric field stimulation in two opposite directions. FIG. 1 exemplary shows an electric pulse sent by controller 112 to terminal 108 with negative polarity and to terminal 109 with positive polarity. These pulses create electric current in right coil 103 in a counterclockwise direction and in left coil 102 in a clockwise direction. In set of parallel wires 105 the electric current, as shown in FIG. 1, is directed downward along the negative direction of the Y-axis, whereas the electric field 114 generated by the set of wires 105, is directed upward along the positive direction of the Y-axis. When a train of electric pulses sent from controller 112 to applicator 101 has a reversed polarity it creates an electric field directed in the negative direction of the Y-axis (not shown in FIG. 1).

In a preferred embodiment, the apparatus for treating atherosclerosis may include two of the planar applicators of FIG. 1 oriented at an angle of 90 degrees to each other. In this configuration, the two-plane applicator will stimulate the treatment zone with the electric field in four directions: in the plus and minus directions along the Y axis and in the plus and minus directions along the X axis.

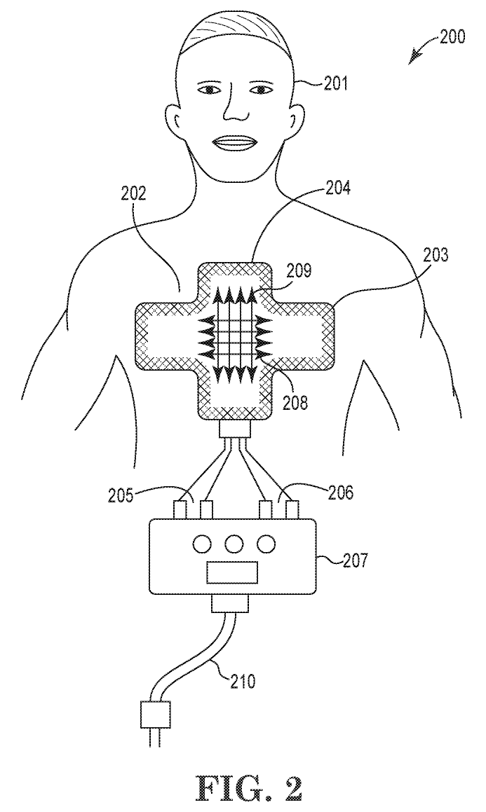

FIG. 2. schematically shows a two-dimensional apparatus for treatment of heart diseases. Number 201 is designated for the patient. The schematically depicted two-dimensional applicator 202, having a flexible fabric cover for its coils, is placed on the patient’s chest close to the heart. The flexible fabric cover of applicator 202 allows it to conform to the patient's body, ensuring close contact and effective delivery of the electric field stimulation.

The two-dimensional applicator 202 comprises a first one-dimensional applicator 203, positioned horizontally and a second one-dimensional applicator 204 positioned vertically. The terminals of the horizontally positioned applicator 203 are connected to the output electrodes 205 of controller 207. This configuration is designed to generate trains of electric pulses of different polarity to provide two directions of electric field stimulation along the X-axis. The terminals of vertically positioned actuator 204 are connected to the output electrodes 206 of controller 207 that separately generate trains of electric pulses of different polarity to provide two directions of electric field stimulation along the Y-axis. The electric field stimulation provided along the X-axis is shown by numeral 208, electric field stimulation provided along the Y-axis is shown by numeral 209. Numeral 210 refers to a power cable that may be connected to a power grid or another power source such as a battery.

In another embodiment, instead of one coil per each of the X and Y directions, each with a bipolar pulse, two parallel coils can be used for both of the X and Y axes. Each of the four coils receives its own monopolar pulse source, generating a single stimulation field per coil. Thus, four-directional stimulation may be achieved using four coils and four monopolar voltage sources.

Additionally, the apparatus disclosed herein is designed to be user-friendly and safe. The power supply is equipped with safety mechanisms to prevent any electrical hazards, and the controller 207 has built-in features to monitor its operation and ensure that the parameters of the electric pulses are within the therapeutic range.

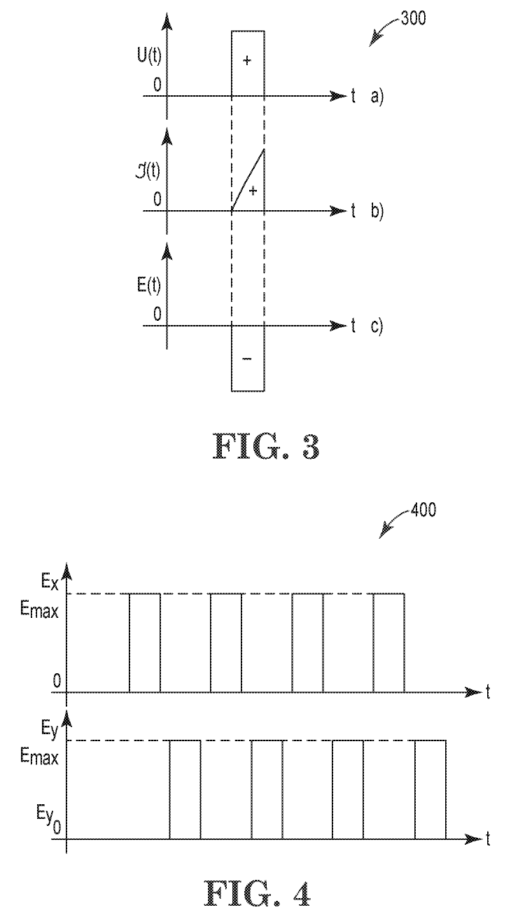

FIG. 3 shows a diagram of a voltage applied to an applicator (a), electrical current and magnetic field in the applicator (b) and the electric field in the treatment zone (c) during operation. The voltage pulse (a) generated by controller 207 on electrodes 205 or electrodes 206 is preferentially rectangular (DC pulse). The graph of the current in an applicator (b) is an exponential curve close to a straight-line. Graph (c) represents a therapeutic electric pulse in the treatment zone.

FIG. 4 illustrates the pulses of electric fields Eₓ and E_{y} in the treatment zone, generated by horizontal and vertical applicators 204 and 203. The pulses alternate at the same frequency without overlapping, which allows for a shorter operation time with equal field exposure. During operation, pulse polarity changes midway to ensure equal stimulation in all four directions: +Eₓ, -Eₓ, +E_{y}, and -E_{y}.

The method for treating atherosclerosis involves exposure of a segment of an artery containing atherosclerotic lesion to stimulation with pulse electric field, carried by PEMF. Requirements for the electric field: the amplitude of the pulses, their frequency, pulse duration and length of treatment should be selected to ensure the translocation of A2aAR receptors from the cytosol to the cell membrane. An exemplary amplitude of electric pulses is 1-20 millivolt/cm, with a duration of 10 - 1000 microseconds. An additional requirement for macroscopic pattern of the stimulating field is the absence of so-called "dead zones", areas in the treatment zone where the amplitude of the electric field is below the required therapeutic minimum and does not produce a therapeutic effect. As for the coronary arteries, stimulation of the entire heart is recommended.

Around the cell the electric field is distributed highly non-uniformly across the membrane, with a maximum at the point where it is normal to the membrane and zero at the cell's "equator" where it is parallel to the membrane. Additional stimulation applied perpendicular to the first direction allows a further boost to A2aAR expression. Therefore, multidirectional stimulation is preferred.

According to Dr. Varani’s data, one-directional stimulation leads to a 120% increase in the expression of A2aARs. Extrapolating this result to four-directional stimulation, it is reasonable to expect at least a 400% increase in the expression of A2aAR receptors and very strong anti-inflammatory effects in atherosclerotic lesions.

In clinical practice, patients may undergo treatment sessions lasting 20-30 minutes, administered 1-2 times daily, for example. Intensive therapy may involve 4-5 sessions with 3-hour intervals, allowing the cellular processes to reset before the next stimulation.

In atherosclerosis, the metabolic processes that maintain a normal state of an artery compete with the destructive effects of inflammation. Thus, the actual state of the atherosclerotic artery is determined by the balance between these two forces. Electrical field stimulation has a direct anti-inflammatory effect on all cells that contribute to inflammation, promoting normal physiology. This leads to strengthening of the fibrous cap on the lesions and enhancement of reverse cholesterol transport (RCT), that clears cholesterol debris and reduces the volume of the lesions, effectively reversing atherosclerosis.

The innovative method of treatment and apparatus, disclosed in current invention, represents a significant advancement in non-invasive anti-inflammatory treatment of atherosclerosis, offering a promising alternative to conventional methods.

While the invention has been described in connection with what is presently considered to be the most practical and preferred example embodiments, it will be apparent to those of ordinary skill in the art that the invention is not to be limited to the disclosed example embodiments. It will be readily apparent to those of ordinary skill in the art that many modifications and equivalent arrangements can be made thereof without departing from the spirit and scope of the present disclosure, such scope to be accorded the broadest interpretation of the appended claims so as to encompass all equivalent structures and products.

It is also within the scope of the invention to combine features, functions, advantages and aspects of the various embodiments described herein. Thus, the embodiments of the invention may comprise combinations of aspects of any one or more of these exemplary embodiments.

For purposes of interpreting the claims for the present invention, it is expressly intended that the provisions of Section 112, sixth paragraph of 35 U.S.C. are not to be invoked unless the specific terms "means for" or "step for" are recited in a claim.

What is claimed is:

1. A method of treating atherosclerosis in a patient, the method comprising:
applying a pulsed electromagnetic field (PEMF) stimulation to a treatment volume within the patient containing an artery of the patient sufficient to increase expression of A2aAR adenosine receptors in immune and parenchymal cells of the patent such that immune system activity of the patient in the treatment volume is reduced.

2. The method of claim 1 wherein the treatment volume includes an entire heart of the patient.

3. The method of claim 1, wherein the PEMF stimulation is applied to the treatment volume in multiple different spatial directions.

4. The method of claim 1, wherein the PEMF stimulation is applied to the treatment volume in a first spatial direction and in a second spatial direction, wherein the second spatial direction is perpendicular to the first spatial direction.

5. The method of claim 1, wherein the treatment volume is free from electrical field dead zones.

6. The method of claim 1, wherein the application of the PEMF stimulation is provided via an applicator applied to a chest of the patient, the applicator including a first coil and a second coil coupled to a controller to create a counterclockwise current in the first coil and a clockwise current in the second coil, wherein the first coil and second coil are arranged to create an electrical field in the treatment volume along a first axial orientation.

7. The method of claim 6, further comprising the controller applying a series of square wave electrical pulses to the first and second coils, wherein the series of square wave electrical pulses are alternatingly positive and negative polarities.

8. The method of claim 6 , wherein the applicator further includes a third coil and a fourth coil coupled to the controller to create a counterclockwise current in the third coil and a clockwise current in the fourth coil, wherein the third coil and fourth coil are arranged to create an electrical field in the treatment volume along a second axial orientation that is perpendicular to the first axial direction.

9. The method of claim 1, wherein the PEMF stimulation is applied with a pulse amplitude of 1-20 millivolt/cm and a pulse duration of 10 - 1000 microseconds.

10. The method of claim 1, wherein the PEMF stimulation is applied to the patient in a first direction and in a second direction perpendicular to the first direction.

11. The method of claim 1, wherein the PEMF stimulation is applied to the patient in multiple sessions per day of 20-30 minutes each session.

12. A method of activating adenosine A2aAR receptors in immune and parenchymal cells involved in inflammation process in atherosclerotic lesions of a patient, the method comprising:
applying a pulsed electromagnetic field (PEMF) stimulation to an atherosclerotic lesion of the patient sufficient to increase expression of A2aAR adenosine receptors in the immune and parenchymal cells of the patent such that inflammation in the atherosclerotic lesion is reduced.

13. The method of claim 12, wherein the PEMF stimulation is applied to the atherosclerotic lesion in multiple different spatial directions.

14. The method of claim 12, wherein the application of the PEMF stimulation is provided via an applicator applied to a chest of the patient, the applicator including a first coil and a second coil coupled to a controller to create a counterclockwise current in the first coil and a clockwise current in the second coil, wherein the first coil and second coil are arranged to create an electrical field in the atherosclerotic lesion along a first axial orientation.

15. The method of claim 14, further comprising the controller applying a series of electrical pulses to the first and second coils with alternatingly positive and negative polarities.

16. The method of claim 14, wherein the applicator further includes a third coil and a fourth coil coupled to the controller to create a counterclockwise current in the third coil and a clockwise current in the fourth coil, wherein the third coil and fourth coil are arranged to create an electrical field in the atherosclerotic lesion along a second axial orientation that is perpendicular to the first axial direction.

17. A pulsed electric field stimulation apparatus for treatment of atherosclerosis within a treatment volume of a patient, the apparatus comprising:
an applicator;
a first coil disposed in the applicator and including a first straight wire segment;
a second coil disposed in the applicator and including a second straight wire segment;
a controller electrically couped to first coil to create a counterclockwise current in the
first coil and electrically coupled to the second coil to create a clockwise current in the second coil,
wherein the first coil and second coil are arranged such that the first straight wire
segment is oriented parallel to the second straight wire segment to create a linear stimulation electrical field along a first axis in the treatment volume.

18. The apparatus of claim 17, wherein the applicator is configured for application to a chest of patient.

19. The apparatus of claim 17, further comprising:
a third coil disposed in the applicator and including a third straight wire segment;
a fourth coil disposed in the applicator and including a fourth straight wire segment;
wherein the controller is electrically couped to third coil to create a counterclockwise
current in the third coil and electrically coupled to the fourth coil to create a clockwise current in the fourth coil,
wherein the third coil and fourth coil are arranged such that the third straight wire
segment is oriented parallel to the fourth straight wire segment to create a linear stimulation electrical field along a second axis in the treatment volume, and
wherein the second axis is oriented perpendicular to the first axis.

20. The apparatus of claim 17, wherein the controller is configured to apply a series of electrical pulses to each of the first and second coils with alternatingly positive and negative polarities.
